(19) United States
(12) Patent Application Publication
(10) Pub. No.: US 2025/0082969 A1
(43) Pub. Date: Mar. 13, 2025

(54) METHODS AND SYSTEMS FOR TREATMENT OF SYSTEMIC INFLAMMATORY DISORDERS

(71) Applicant: The Board of Trustees of The Leland Stanford Junior University, Stanford, CA (US)

(72) Inventors: Nishant DOCTOR, Stanford, CA (US); Alexander SACKEIM, Stanford, CA (US)

(73) Assignee: The Board of Trustees of The Leland Stanford Junior University, Stanford, CA (US)

(21) Appl. No.: 18/960,847

(22) Filed: Nov. 26, 2024

Related U.S. Application Data
(63) Continuation of application No. PCT/US2023/024133, filed on Jun. 1, 2023.
(60) Provisional application No. 63/347,860, filed on Jun. 1, 2022.

(51) Int. Cl. A61N 7/00

(52) U.S. Cl. CPC A61N 7/00; A61N 2007/0026

(57) ABSTRACT

Medical energy devices, systems, and methods that allows for treatment of systemic inflammatory disorders through modulation of neural activity.

### CROSS-REFERENCE TO RELATED APPLICATIONS

This application is a continuation of PCT/US2023/024133 filed on Jun. 1, 2023, which claims the benefit of priority to U.S. provisional application No. 63/347,860 filed Jun. 1, 2022, the entirety of both of which is incorporated by reference.

### FIELD OF THE INVENTION

The embodiments of the current invention relate to medical technology that allows for treatment of systemic inflammatory disorders through modulation of neural activity using electronics.

### BACKGROUND OF THE INVENTION

The innate immune system plays an important role in human immunity. Without it, humans would be much more susceptible to a variety of pathogens. However, an overactive innate immune system is also central to a variety of human diseases. These include autoimmune diseases such as Rheumatoid Arthritis, Chron's disease, and Ulcerative Colitis, as well as infectious disease when those infections result in systemic inflammatory response syndrome (SIRS) or septic shock. The innate immune system can also worsen a variety of other diseases including metabolic disorders such as obesity and Type 2 diabetes, cardiovascular disease, or ischemic reperfusion injuries in organ transplant or hypoperfusion. The underlying mechanisms of each of these disease processes are different, but the unifying factor is that pro-inflammatory cytokines are released by monocytes, macrophages, and dendritic cells that create a positive feedback loop leading to greater systemic inflammation. These cytokines and chemokines include but are not limited to, TNF-alpha, IL-1b, IL-6, IL-8, IL-12, IL-18, IFN-gamma, and HMGB1.

In the case of Rheumatoid Arthritis (RA), the immune system recognizes proteins in the synovium as foreign, causing inflammation in the joints and surrounding tissues, which slowly destroys the joints and tissues. This process is amplified by the release of pro-inflammatory cytokines by innate immune cells, which in turn leads to further joint destruction. This destruction of cartilage and bone causes pain, discomfort, and stiffness, and the condition is estimated to affect a large number of adults every year, with a current prevalence of about 1.3 million people in the United States. RA carries a substantial disease burden, causing pain, disability, and impaired health-related quality of life. RA patients develop work disability that can cost them their jobs. Studies report that approximately 35-50% of RA patients stopped participating in certain activities, with about 30% reported retiring from work, quitting a job, or switching jobs as a result of the disease. Beyond the patient impact, the disease is estimated to cost the US healthcare system $19.3 billion annually. Some of the most expensive medications for private and public payers are for the treatment of RA.

Pharmaceuticals are the common treatment for symptomatic RA patients where clinical guidelines define the goal of RA treatment to be remission or low disease activity if remission is not possible. Currently, there are two major medication treatment options to meet these goals, 1) conventional synthetic DMARDs (csDMARD), or 2) targeted biologic (bDMARD) or targeted synthetic DMARDs (tsDMARD). While studies have shown that these targeted therapies result in improved clinical outcomes and a reduction in disability, 20-30% of the RA patients do not achieve meaningful clinical benefit and response to the therapy. Of the patients who respond to therapy, studies have shown that approximately half of the patients remain on the initial therapy after 5 years. Surveys indicate that treatment side effects and the number/frequency of medications are the top reasons to discontinue the existing therapies. Roughly, about 60% or more of RA patients are taking pain relief medications, along with their DMARDs, to help manage their disease.

Many of the treatments for systemic chronic inflammatory disorders have significant shortcomings. Paramount among these is lack of efficacy. Approximately 50% of patients who fail first-line treatment for RA with Methotrexate respond to a TNF-a blocker. Furthermore, these medications can cause adverse effects such as rashes, headaches, nausea, and joint pains. In some cases, these reactions can be severe, causing hypotension, anaphylaxis, or severe infection.

In view of these shortcomings with the current standard of care for RA, there remains a need for an alternative noninvasive neuromodulation therapy that can treat chronic inflammatory disorders that improve the current standard of care to improve efficacy and reduce side effects in patients.

### SUMMARY OF THE INVENTION

The present disclosure includes methods of applying an energy to one or more target nerves within a patient. For example, a variation of such a method includes providing an energy device having a marker, where the marker is detachable from the energy device and where the energy device comprises an nesting area permitting coupling of the marker and the energy device; determining an area on an exterior surface of the patient that allows passage of energy from the energy device through the exterior surface to the one or more target nerves; affixing the marker on the area at the exterior surface; coupling the energy device to the marker and to the patient such that the marker aligns the energy device towards the one or more target nerves; applying energy from the energy device to the one or more target nerves through the exterior surface of the patient; and detaching the energy device from the marker and the patient, where the marker remains affixed to the patient to permit subsequent attachment of the energy device to the marker and the patient and where the marker permits alignment of the energy device to the one or more target nerves.

A variation of the method includes determining the area on the exterior surface of the patient by noninvasive imaging of the patient to identify a target area containing the one or more target nerves. Alternatively, the determination can occur by palpitation or application of energy until a sufficient effect is achieved.

Variations of the methods and systems include the use of ultrasound energy as the therapeutic energy.

The markers described herein can comprise radio frequency identification (RFID) sensors and electrical sensors. In addition, the markers can also be magnetically coupled to the energy device. The magnetic features of the marker and device can provide a fail safe such that the energy device is configured activate only when the marker is coupled to the energy device.

In additional variations of the device, the energy device is configured to monitor a respiration cycle of the patient. Moreover, where the energy device can include one or more actuators to reposition the energy device in response to movement of the patient.

Additional variations of the methods and devices include applying energy from the energy device by applying a series of energy treatments to stimulate the one or more target nerves and varying a dosing parameter of one or more of the series of energy treatments to prevent nerve adaption of the one or more target nerves. Varying the dosing parameter can include altering a frequency of one or more of the series of energy treatments in a fixed step rate or a random step rate manner. In addition, varying the dosing parameter can include altering frequency of one or more of the series of energy treatments.

Another variation of the method can include performing a repeat therapy to treat one or more target nerves with energy, where a marker is affixed to a patient such that the marker is previously positioned on an exterior surface of the patient in alignment with the one or more target nerves within the patient. For example, such a method can include coupling an energy device to the marker such that the marker aligns the energy device with the one or more target nerves; applying energy from the energy device to the one or more target nerves through the exterior surface of the patient; and detaching the energy device from the marker and the patient, where the marker remains affixed to the patient to permit subsequent attachment of the energy device to the marker and the patient and where the marker permits alignment of the energy device to the one or more target nerves.

The present disclosure also includes systems for providing a repeated therapy to an internal organ within a patient. In one example, the system comprises a marker configured to be affixed to an exterior of the patient; and an energy device having an energy treatment portion and having a fastener configured to permit securing of the energy device to an exterior of the patient, the energy device further including a mating surface to couple the marker and the energy device, such that when the marker is affixed to the exterior of the patient the energy device can be coupled to the marker allowing for automatic alignment of the energy treatment portion to the internal organ from the exterior of the patient.

The marker can include an adhesive for affixing to the exterior of the patient or one or more suture openings for affixing to the exterior of the patient using a suture.

As noted above, the system can include one or more actuators to reposition the energy device in response to movement of the patient. Such actuators can be controlled independently or by the controller.

In an additional variation, the energy device includes a controller that is configured to apply a series of energy treatments to stimulate the one or more target nerves and varies a dosing parameter of one or more of the series of energy treatments to prevent nerve adaption of the one or more target nerves. The controller can alter a frequency of one or more of the series of energy treatments in a step rate manner or in a random manner. The controller can also be configured to vary the dosing parameter by altering an intensity of one or more of the series of energy treatments.

As noted herein, for the system to be user-friendly and easy to accurately apply over the target region, at home by an average user, the product and directions/guidance for use need to be simple and easy to understand. The system would also monitor the movement of the spleen using imaging or building complex AI models for detection.

As discussed herein, the systems and methods provide a therapeutic ultrasound device that modulates the activity of neurons, nerves, and nerve plexuses, specifically, but not limited to neurons in the vagus nerve and splenic nerve that make up the Cholinergic Anti-Inflammatory Pathway.

### BRIEF DESCRIPTION OF THE FIGURES

FIG. 1 provides an illustration of applying energy to a nerve fiber to affect cholinergic anti-inflammatory pathway.

FIG. 2A provides an illustration of a patient and an approximate location of the spleen.

FIG. 2B is an illustration to showing an energy device applying a therapeutic energy treatment exterior to the patient to produce a neural response in a splenic nerve.

FIG. 2C shows a spleen located behind ribs of the ribcage.

FIG. 3 illustrates a back view of a patient and shows a position of the spleen with a marker attached to a surface of the patient.

FIG. 4A illustrates an example of an energy device configured to provide therapy as described herein.

FIG. 4B illustrates an alternate example of an energy device configured to provide therapy as described herein.

FIG. 4C illustrates the energy device coupled to a patient.

FIGS. 5A and 5B illustrate steering of the unit to account for movement of the patient while still delivering therapy to the targeted nerve.

FIG. 6A illustrates a burst treatment to reduce nerve adaption.

FIG. 6B shows an example of a single transducer delivering output pressure.

FIG. 7 illustrates one example of a system block diagram for the system described herein.

FIGS. 8A to 8C illustrate three different sensor variations to continuously monitor cytokine levels using electrochemical immunosensors.

### DETAILED DESCRIPTION

The methods, systems, and devices described herein can provide efficacy in treatment by using the cholinergic anti-inflammatory pathway (CAP), which regulates the innate immune response to injury, pathogens, and tissue ischemia. This pathway is the efferent, or motor arm of the inflammatory reflex, which is the neural circuit that responds to and regulates the inflammatory response.

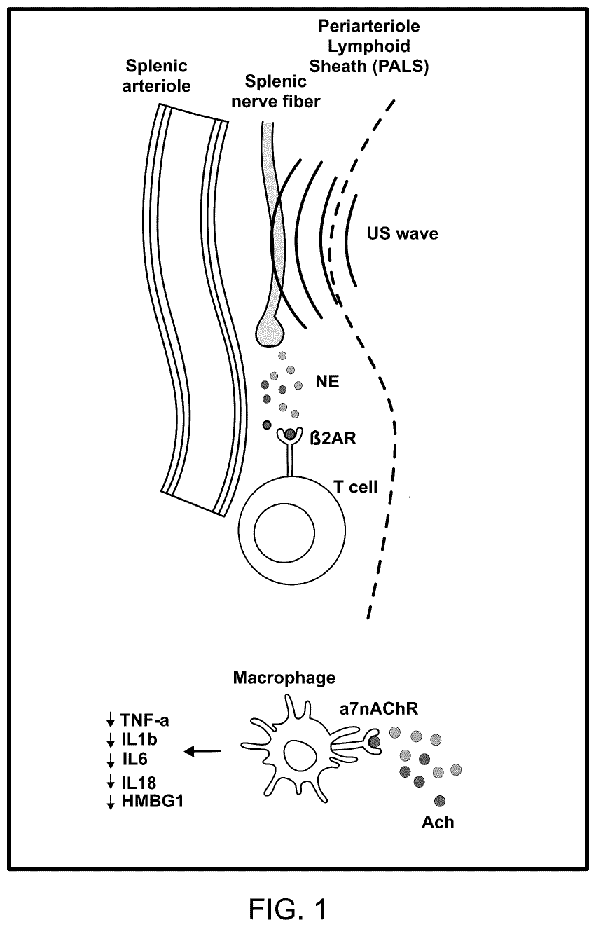

It has been demonstrated in humans that stimulating efferent fibers of the vagus nerve can lead to an anti-inflammatory effect. Further studies using electrical nerve stimulation, genetic knock-outs, and pharmacologic antagonists have isolated the associated afferent and efferent nerve pathways. The afferent limb has inputs from multiple organs via vagal paraganglia as well as the auricular branch of the vagus nerve. These neurons pass through the nodose ganglia where their cell bodies lie and synapse in the solitary nucleus (NTS). There is some afferent signaling as well via the sciatic nerve. The efferent limb begins in the dorsal motor nucleus (DMN), with its cell bodies lying in the nodose ganglia, after which it synapses in the celiac ganglia in the abdomen. The post-synaptic branch is the splenic nerve which follows the splenic artery and its branches into the spleen. Here the splenic nerve fibers synapse on T-cells located in the periarterial lymphoid sheath (PALS). Here local binding of norepinephrine and epinephrine with adrenergic receptors on T-cells leads to the release of acetylcholine. This binds to a 7-nicotinic acetylcholine receptor on monocytes and macrophages, which in turn downregulates the release of pro-inflammatory cytokines by these cells, as illustrated in FIG. 1.

The present disclosure includes variations of methods, systems, and devices that modulate the activity of the vagus nerve to have an excitatory or inhibitory effect based on the parameters used. However, in one variation involving the CAP, these technologies excite targeted nerves. While different variations of the treatment described herein can include energy from different sources, including ultrasound, electromagnetism, transcutaneous electricity, infrared light, and a combination thereof. as detailed below. While the different energy modalities excite neurons in different ways based on the technology. The effect of more frequent depolarizations and the release of specific neurotransmitters leading to a decrease in the pro-inflammatory response of innate immune cells is the same.

In one variation, the application of ultrasound energy modulates neural activity along the CAP. The variation optimizes the number and shape of the transducers, the acoustic energy intensity (both positive and negative acoustic pressure), focal zone, acoustic wave frequency, pulse duration, pulse repetition frequency (PRF), and treatment duration in order to activate the cholinergic anti-inflammatory response.

A variation of a method under the present disclosure includes a noninvasive or transcutaneous approach to modulate the activity of nerves and immune cells in the cholinergic anti-inflammatory pathway. The treatment can target anywhere along this pathway, including, but not limited to, the NTS or DMN of the medulla, the superior ganglia (SG), the nodose ganglia (NG), the auricular branches of the vagus nerve (ABVN), the cervical vagus nerves (CVN), the celiac ganglion (CG), the splenic nerve (SN), and the splenic nerve fibers and their end terminals in the spleen, nerve fibers and their end terminals in the intestines and pancreases. These targets may include afferent or efferent branches of the vagus nerve. Two or more targets specified above can be stimulated at the same time or in sequential order to enhance the end outcomes effects. For example, methods and devices can stimulate the spleen and the intestine at the same time or in sequential, that is, stimulate the spleen first and then the intestine, or vice versa.

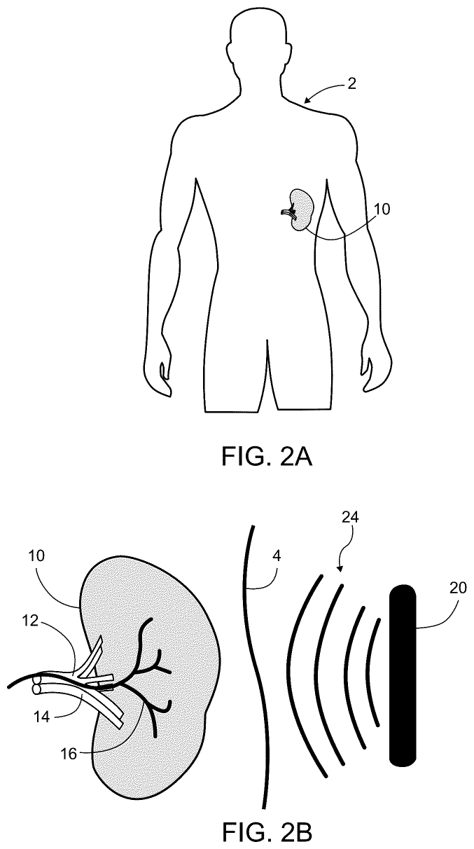

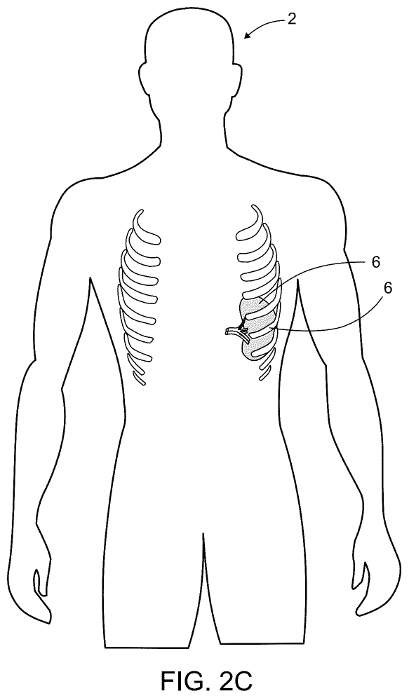

A variation of the treatment targets the splenic nerve for innervation of the spleen. Targeting of the splenic nerve focally targets the efferent signaling of the CAP without stimulating other efferent branches of the vagus nerve, which can result in off-target effects in other organs such as the muscles in the throat, the heart, lungs, liver, stomach, and intestines. The location of the spleen is also a fairly accessible target for noninvasive neuromodulation. FIG. 2A provides an illustration of a patient 2 and an approximate location of the spleen 10, which is located in the retroperitoneum. The spleen 10 is often only 1-2 centimeters from the skin surface. However, when at rest after exhalation, the spleen 10 is usually tucked beneath the left rib cage superolateral to the left kidney and posterolateral to the stomach. The superior pole is often obscured posterior by the inferior lobe of the left lung.

FIG. 2B provides an illustration to convey an energy device 20 applying a therapeutic energy treatment 24 across a surface 4 of the patient to produce a neural response in the splenic nerve 16, which generally runs along the splenic artery 14 and splenic vein 14.

It was found that when using ultrasound energy, the positive pressure, also referred to as the compressional pressure or acoustic radiating force is key to generating a neural response, and pressure levels below a certain threshold do not appear to generate depolarization of neurons. In one variation, the threshold pressure to trigger a response in the focal zone was between 100-200 kPa (2-3× atmospheric pressure), but higher pressures tend to recruit more neurons and trigger a larger response. However, positive pressures that exceed a certain pressure threshold can also be deleterious. The higher the pressure, both positive (compression) and negative (rarefaction), the more likely there will be tissue injury of some kind. Human tissues appear to tolerate higher positive pressure thresholds up to about 15 MPa as long as they are very short pulse durations and low pulse repetition frequencies. On the other hand, negative pressures must be lower, usually not to exceed 4 MPa, to avoid cavitation and the resultant tissue injury. This is particularly true at ultrasound frequencies less than 1 MHz. However, the present disclosure can include any range that produces the desired therapeutic effect.

The focal zone for ultrasound treatment is the three-dimensional space where the ultrasound field is at least ½ the peak spatial-temporal positive pressure (MPa) (e.g., as measured with a hydrophone in a degassed water bath). For example, if the spatial-temporal peak is 3 MPa then the focal area is anywhere where the spatial temporal peak is at least 1.5 MPa. For a smaller focus, it is possible to use a concave transducer design that creates a cone shaped field with a focal zone from 1-10 cm\hat{ }3. For a larger focus, the device can use a planar design that creates a columnar or cuboidal shaped field depending on the transducer, and has a focal zone of 20-150 cm\hat{ }3.

The pulse duration of the ultrasound treatment can be as short as 1 microsecond and as long as 1 second or be continuous. In one variation, a pulse duration is several hundred microseconds, as this appears to balance the need for successive positive pressure waves to elicit a response while balancing the time-averaged intensity of the ultrasound beam and the associated tissue heating. The PRF can range from <1 Hz to greater than 1 kHz. However, it is most likely to be in the 1-10 Hz range as this tends to be stimulatory to neurons.

The treatment duration can range from less than 1 minute to more than 1 hour. It may even be a continuous therapy to be applied throughout the day. However, the likely treatment duration will be dictated by the need to achieve the desired effect while being comfortable and usable for the patient.

In order to perform successive sessions of noninvasive ultrasound neuromodulation therapy, there is a need for the energy device to be removed from the patient after a session and then reattached to the patient. If the treatment is performed at the patient's home, the patient (or caregiver) must be able to position the device against the exact anatomical structure being targeted. However, as shown in FIG. 2C, the spleen 10 is located behind ribs 6 of the ribcage. Most untrained individuals do not know how to locate the target structures in their body or where to locate the treatment device on an exterior surface of the body. To complicate matters, some of the structures targeted for treatment, such as the spleen 10, can move several centimeters with respiration and/or a change in body position. Even with training using external landmarks, like rib counting, it can be hard to accurately locate the spleen. Therefore, improper placement of the energy device can reduce effectiveness of the therapy or produce other undesirable effects.

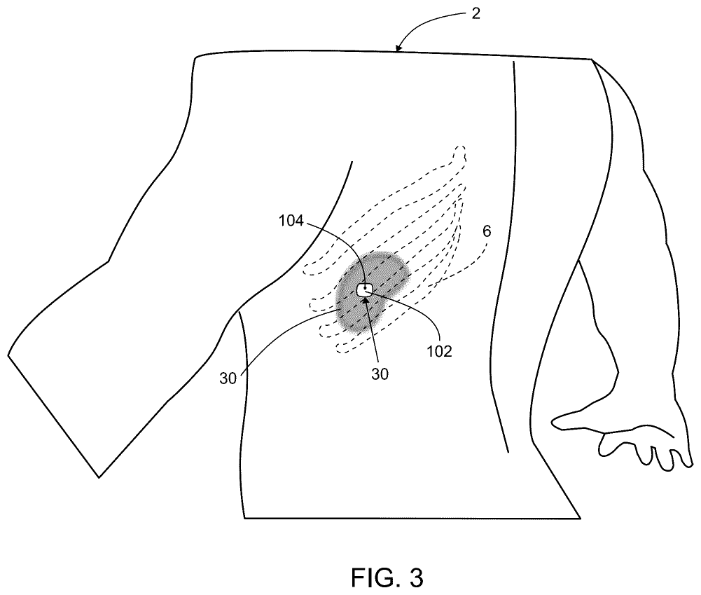

To address these challenges, the methods and systems of the present disclosure allow for a trained medical caregiver to locate the spleen (or other targeted nerve area) and locate an area 30 on an exterior of the patient 2 that will be sufficient for applying the therapy. FIG. 3 illustrates a back view of a patient 2 to show the spleen 10, which is typically between 8-12 ribs on the left side. The caregiver can locate the area 30 on the surface of the patient using any number of methods. For example, the caregiver can feel for the various ribs to identify the area 30. Alternatively, the caregiver 30 can rely on any noninvasive imaging such as X-ray, regular ultrasound diagnostic equipment, etc. In another variation, the caregiver can position the device (as described below) and apply therapeutic treatment to determine proper positioning of the device.

Once the caregiver identifies an acceptable area 30, the caregiver can attach a biocompatible marker 102 on the area 30, as shown in FIG. 3. This marker 102 can include any number of attachment features 104 such as adhesive, tape, suture openings (for temporary suturing of the marker 102 to the individual. Precise location that can be visualized by anyone.

The marker 102 can comprise an electromagnetic device (“sensing and guidance device”) as small as size of two to three stacked penny, dime, or quarter. The marker 102 can be placed on the middle region of the highlighted spleen 30, such that it sits, but not limited to, directly over the hilum of the spleen 30, on the skin. In some variations, the attachment feature permits the marker 102 to remain affixed to the patient 2 for up to 90 days. It is noted that the caregiver can also temporarily mark the area 30 on which the marker is placed. The marker 102 can comprise an RFID, or electrical sensor, or a simple magnet.

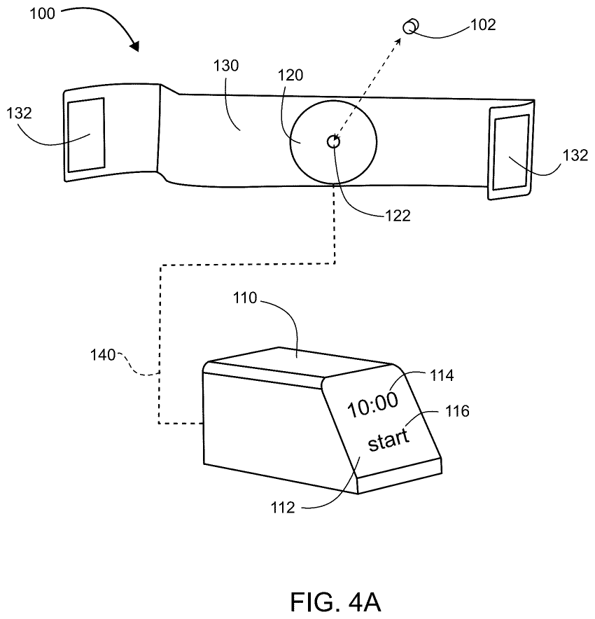

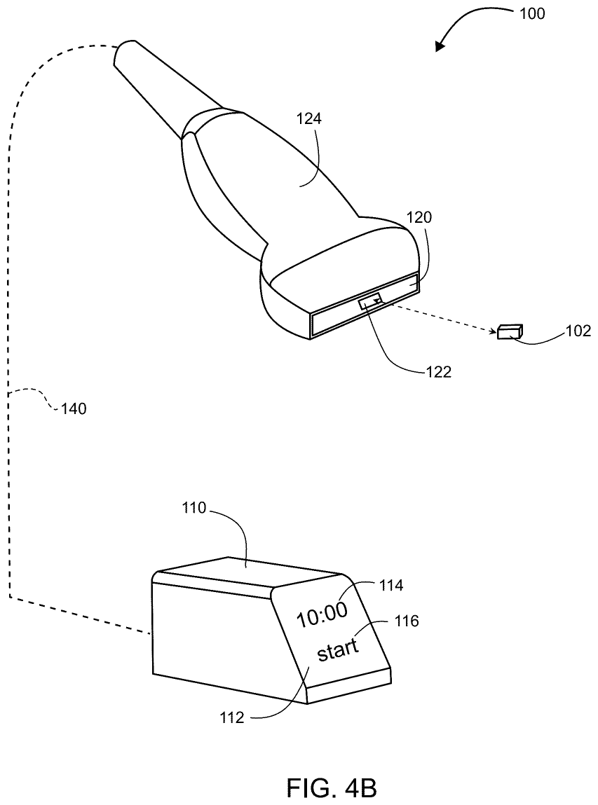

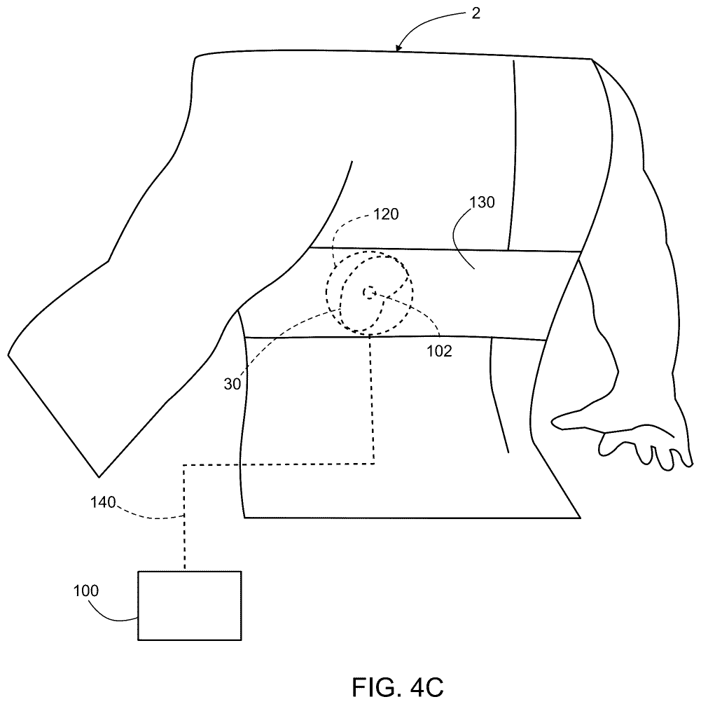

FIG. 4A illustrates an example of an energy device 100 configured to provide therapy as described herein. In this variation, the device 100 includes an ultrasound transducer 120 coupled to a fastener or fixation structure 130 (e.g., a strap, belt, elastic band, etc.) that is configured to secure the energy device to an exterior of the patient. In the illustrated variation, the fastener 130 includes one or more regions 132 (e.g., adhesive regions, hook-and-loop, magnets, etc.) that allow the device 100 to be secured to a patient. In addition, the energy device 100 includes a marker 102, as described above. The marker 102 is configured to be affixed to an exterior of the patient (e.g., via an adhesive, suture, or other temporary fixation means) and is also configured to couple to a mating surface 122 on the device 100. In the illustrated variation, the mating surface 122 is located in the ultrasound transducer assembly 120 such that when the transducer assembly 120 and marker 102 are coupled when the marker is affixed to a patient, the transducer assembly 120 is automatically oriented towards a target nerve region within the patient. In another variation, the marker 102 can be coupled adjacent to the transducer 120 (e.g., on the fastener 130). In one variation, the ultrasound transducer 120 comprises a non-imaging therapeutic ultrasound transducer. Some variations of the transducer 120 allow for the mating surface 122 to comprise an opening in the transducer 120 to allow seating of the marker 102 within the opening. In additional variations, the marker 102 is configured to permit ultrasound energy to pass through to the tissue.

The energy device 100 further includes a controller 110 that controls delivery of energy as described herein. In the illustrated variation, the controller 110 also includes a display screen 112 that can allow an individual to control the energy delivery device 100. In the illustrated variation, the display screen includes treatment time information 114 and a touch screen to initiate the treatment 116. In the variation shown in FIG. 4A, the controller is coupled to the transducer 120 via a cable (e.g., for an ultrasound energy modality). However, in alternate variations, the controller 110 can control energy delivery wirelessly.

The marker 102 acts as detection mechanism and, in some variations, can send a signal to the wearable ultrasound transducer 120 for easy snap and fit of the therapeutic transducer over the electromagnetic device. The sensing and guidance device can also serve two additional purposes. The marker 102 can act as a navigation/guidance tool for accurate identification/landmark of the targeted nerve and assist the user to accurately place the therapeutic device over the target site once the marker is properly positioned 102. Therefore, coupling the transducer (or other energy delivery modality) can reduce the chances of prevent any off-target therapy delivery that would result in lower effectiveness of the therapy or result in the application of energy to an unintended target area.

Additionally, the marker 102 can provide a sensing function that allows activation of the therapeutic device 100 only when the marker 102 is properly seated in a receiving area 122. This receiving area 122 can comprise a pocket, cavity, or other structure that allows a user to confirm positioning of the transducer 120 (or other part of the device 100) with the marker 102, where the marker 102 is previously positioned on the patient to direct energy towards the targeted nerves. In such variations, without accurate placement of the therapeutic transducer 120 over the marker 102, the energy device 100 will not activate or deliver the therapy. This is a fail-safe mechanism to avoid the user from delivering therapy to unintended targets by wearing the therapeutic device upside down or higher or below the intended area.

The sensing circuitry installed within the marker 102 can be an RFID circuit or a plain electrical circuit with contact touch points that are electrically charged when connected to the electrical touch points on the therapeutic device. When they come in contact, it allows for activation of the therapeutic device 100 for delivery of therapy. In an additional variation, the marker 102 and/or receiving area 122 can be magnetic (either or both components can be magnetic) such that the device 100 and/or controller can include a magnetic sensor that determines magnetic coupling of the marker 102 and transducer 120 prior to allowing for activation of the energy device 100.

FIG. 4B shows another variation of an energy device 100 where the device includes an ultrasound probe array 120 on a traditional ultrasound device 124 coupled to a controller 1010 via a probe or other common means. As shown, the ultrasound surface 120 includes a receiving feature 122 (as described above) that allows for coupling of a marker 102. The variations of the marker 102 and receiving feature 122 that apply to FIG. 4B include the combinations described with respect to FIG. 4A. The probe 124 can be held in place by the user to deliver therapy as long as the marker 102 and receiving feature 122 are sufficiently coupled, as discussed above. Alternatively, the probe 124 can be incorporated into a belt or strap (not shown) to hold the transducer assembly 120 in engagement with the patient as long as the marker 102 is coupled to the receiving feature.

FIG. 4C illustrates the energy device coupled to a patient 2. As shown, the transducer 120 and marker 102 are coupled together while the marker 102 remains affixed to the patient 2 over the region of interest (in this variation, the spleen 30). The fastener or band 130 permits the transducer 120 to remain on the patient 2 during treatment.

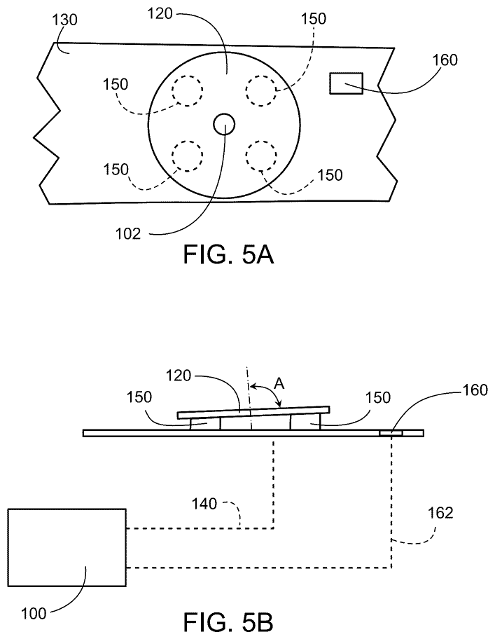

FIGS. 5A and 5B illustrate an additional variation of a device that include a sensing capability to monitor a respiration cycle of the patient. As mentioned earlier, the spleen moves 1-4 cm with each breath depending on shallow vs deep breathing. To account for these variations and ensure sufficient therapeutic energy is targeted at the targeted nerve when the therapeutic device is placed over the desired area, the transducer 120 can include one or more actuators 150 that allow “steering” of the transducer 120. The system can further include one or more inertial or displacement sensors 160 that can monitor the patient for movement during breathing. For example, the system can be mechanically steered (i.e. moved an amount shown as angle A) between 1-4 cm in the direction of the spleen movement using the mechanical actuator 150. While the illustration of FIG. 5A shows four actuators 150, any number of actuators can be installed within the therapeutic device. Moreover, the actuators and/or sensor 160 can relay information back to the controller such that the controller determines movement of the system.

In an additional variation, mechanical steering can occur in sync with the respiration cycle that is detected by the respiration monitor 160. For example, during inhalation the spleen moves 1-3 cm downwards, and therefore the transducer face will need to tilt by similar degrees to account for these changes whose direction of movement and intensity of movement will be detected using a standard respiration monitor.

The steering does not necessarily have to be tied to the respiration monitor 160. A person's typical breathing pattern can be calibrated within the system and programmed to steer the ultrasound transducer face by certain degrees at a set interval based on that pattern.

It is understood that the inventions described herein can be embodied in many different forms. The present disclosure is to be considered as an exemplification of the principles of the invention and is not intended to limit the invention to the specific embodiments illustrated.

The methods and systems of the present disclosure can also address the problems of “nerve adaptation” that result from treatment. Nerve adaption occurs when the neuronal activity starts to decay over time in response to repeated stimulation. When this occurs, the nerve response to stimulation becomes erratic or ceases. This frequently occurs on the drug side as well, where the patient's body adapts to a drug and efficacy drops. In this scenario, the user changes the drug or its dose, or in the case of electrical neuromodulation, they manually reprogram the stimulation dosing parameters of the device every few weeks to months to new sets of parameters, and the neuronal activity restores. The reprogramming or dose change typically does not occur until the patient's symptoms reappear, and that acts as a feedback loop to consider changing the dosage.

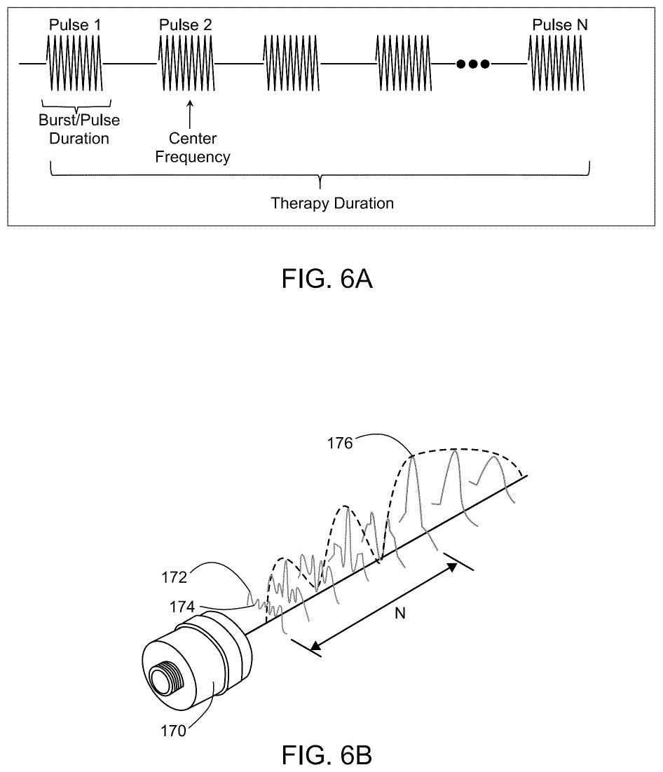

To address nerve adaption, variations of the present disclosure employ automatic alternating pulse/burst pattern of the ultrasound parameters to prevent “nerve adaptation,” as shown in FIG. 6A: Each ultrasound transducer within the therapeutic device delivers each burst/pulse of stimulation at a different ultrasound center frequency in an incremental stepwise fashion at a fixed or unfixed value. For example, if the ultrasound stimulation needs to be delivered, but not limited to, between 500-800 Khz center frequency, then the therapeutic device will sweep from 500-800 Khz in 20 Khz steps every other burst. It will cycle through that range in those increment steps during the duration of therapy. The step size can be programmable at a fixed or random step rate within the given range. Additionally, each burst duration, pulsed frequency, and intensity can also be made programmable to increase or decrease within a range at a fixed or random rate. The alternation can occur at every burst or every other or every third etc., or after each therapy.

The alternative pulsing pattern described above can be achieved using a single wideband therapeutic transducer or using multi-array transducers that are configured at different center frequencies and allowed to delivering alternating pulse/burst pattern within each element as well as across elements. As represented in FIG. 6B, there is an added benefit to above subsequent alternative burst pattern in case of a single-element planar transducer 170. When a single-element planar transducer 170 delivers acoustic energy at a fixed center frequency, there is high acoustic intensity variation within the near-field N, i.e., field close to the transducer face. The field has high number of peaks 172 and nulls 184 of intensity along with the width of the beam, instead of a uniform normal distribution 176 type intensity beam spread that occurs in the far-field as seen in the figure. These peaks and nulls in the near-field can make the ultrasound therapy less effective and potentially unsafe. The alternating pulse/burst pattern is novel approach to address this issue in the near-field where spatially every point in the beam spread has a more uniform beam intensity spread.

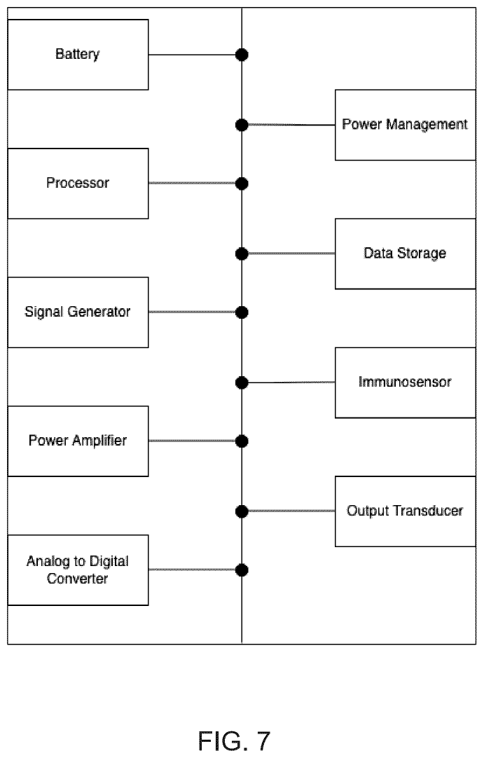

FIG. 7 illustrates one example of a system block diagram for the system described herein. The signal generator outputs the electrical signal to the transducer that converts that signal to acoustic energy. The generator outputs a signal of center frequency that can range from 100 Khz to 60 Mhz, however, based on known experiments, a frequency within the range of 800 Kz-5 Mhz, and 30-50 Mhz have shown be most effective to depolarize the nerve fibers and generate an action potential with maximum efficacy. The generator can modulate the output frequency by delivering the burst of pulses with a duration from range of 1 uS to 100 mS and a pulse repetition frequency (PRF) in range of 1 Hz to 3 Khz. The power amplifier identified in FIG. 7 can deliver an output signal voltage that is necessary to deliver an output pressure from the transducer within range of 100 KPa to 15 MPa positive peak pressure. The signal generated by the generator can be a continuous sinusoidal pulses of a single frequency in the range of 100 Khz to 50 Mhz with a burst duration in range of 1 uS to 100 mS and PRF from 1 Hz to 3 Khz), or a combination of two sinusoidal frequency where one burst of continuous pulses of a one center frequency and output energy followed by another burst of continuous pulses of a different center frequency and output energy with a different duration of burst and PRF (See FIG. 6B). The burst of sinusoidal pulses can have the same symmetrical positive and negative amplitude, or an asymmetrical higher positive peak amplitude with a smaller negative peak amplitude. This asymmetrical waveform can help with reducing the cavitation effect, and increase the mechanical effect of the therapy.

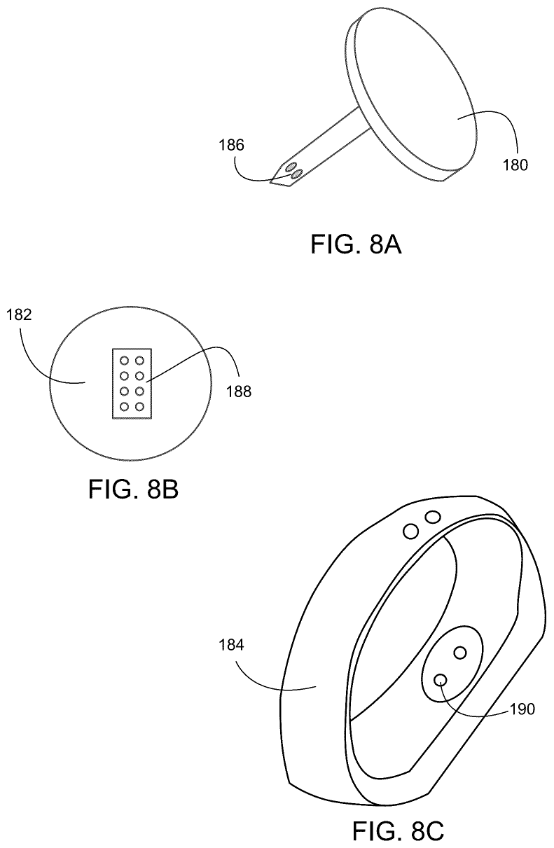

FIGS. 8A to 8C illustrate three different sensor variations, 180, 182, 184 to continuously monitor cytokine levels using electrochemical immunosensors. FIG. 8A has a sensor strip 186 on the main unit 180 that can be delivered into the blood vessel or interstitial fluid to perform cytokine measurement. FIG. 8B is a sensor 182 that is noninvasive and has an immunosensor 188 that can monitor cytokine levels through saliva, urine or sweat. This sensor can be wearable or non-wearable device. In the non-wearable version, the measurement can be performed by placing small quantities of the samples on the immunosensor transducer. These immunosensors devices use immunochemical reactions that are coupled with appropriate transducer, such as, but not limited to, potentiometric, impedance, conductometric etc., that provide the electrical signal intensity that is correlated to the levels of cytokines. FIG. 8C is a wearable sensor 184 that uses optical sensors 190 that emit light of certain wavelength and looking for the amount of reflections of those wavelengths through a detector. The building blocks for all these embodiments are included in FIG. 7 of an overall system block diagram. This continuous monitoring of cytokine levels will act as a feedback loop to the therapy generator and adjust the stimulation parameters depending on the detection levels of cytokines. A closed-loop system is critical for efficacious treatment and ensuring that the therapy dose levels are adjusted based on the fluctuations of the pro-inflammatory cytokine levels.

As for other details of the present invention, materials and manufacturing techniques may be employed within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item includes the possibility that there is plural of the same items present. More specifically, as used herein and in the appended claims, the singular forms “a,” “and,” “said,” and “the” include plural references unless the context clearly dictates otherwise.

It is important to note that where possible, aspects of the various described embodiments or the embodiments themselves can be combined. Where such combinations are intended to be within the scope of this disclosure.

1. A method of applying an energy to one or more target nerves within a patient, the method comprising:
providing an energy device having a marker, where the marker is detachable from the energy device and where the energy device comprises an nesting area permitting coupling of the marker and the energy device;
determining an area on an exterior surface of the patient that allows passage of energy from the energy device through the exterior surface to the one or more target nerves;
affixing the marker on the area at the exterior surface;
coupling the energy device to the marker and to the patient such that the marker aligns the energy device towards the one or more target nerves;
applying energy from the energy device to the one or more target nerves through the exterior surface of the patient; and
detaching the energy device from the marker and the patient, where the marker remains affixed to the patient to permit subsequent attachment of the energy device to the marker and the patient and where the marker permits alignment of the energy device to the one or more target nerves.

2. The method of claim 1, wherein determining the area on the exterior surface of the patient comprises noninvasive imaging of the patient to identify a target area containing the one or more target nerves.

3. The method of claim 1, wherein the energy device comprises an ultrasound energy.

4. The method of claim 1, wherein the marker comprises a sensor selected from the group consisting of a radio frequency identification (RFID) sensor and an electrical sensor.

5. The method of claim 1, wherein the marker is configured to be magnetically coupled to the energy device.

6. The method of claim 1, wherein the energy device is configured activate only when the marker is coupled to the energy device.

7. The method of claim 1, wherein the one or more target nerves comprise a nerve selected from a group consisting of a superior ganglia of a vagus nerve, an inferior ganglia of the vagus nerve, a cervical vagus nerve, a thoracic vagus nerve, an abdominal vagus nerve, a celiac plexus nerve, a splenic nerve.

8. The method of claim 1, where the energy device is configured to monitor a respiration cycle of the patient.

9. The method of claim 1, where the energy device comprises one or more actuators to reposition the energy device in response to movement of the patient.

10. The method of claim 1, where applying energy from the energy device comprises applying a series of energy treatments to stimulate the one or more target nerves and varying a dosing parameter of one or more of the series of energy treatments to prevent nerve adaption of the one or more target nerves.

11. The method of claim 10, wherein varying the dosing parameter comprises altering a frequency of one or more of the series of energy treatments.

12. The method of claim 11, wherein altering the frequency comprises altering the frequency in a fixed step rate or a random step rate.

13. The method of claim 10, wherein varying the dosing parameter comprises altering frequency of one or more of the series of energy treatments.

14. A method of performing a repeat therapy to treat one or more target nerves with energy, where a marker is affixed to a patient such that the marker is previously positioned on an exterior surface of the patient in alignment with the one or more target nerves within the patient, the method comprising:
coupling an energy device to the marker such that the marker aligns the energy device with the one or more target nerves;
applying energy from the energy device to the one or more target nerves through the exterior surface of the patient; and
detaching the energy device from the marker and the patient, where the marker remains affixed to the patient to permit subsequent attachment of the energy device to the marker and the patient and where the marker permits alignment of the energy device to the one or more target nerves.

15. The method of claim 14, wherein the energy device comprises an ultrasound energy.

16. The method of claim 14, wherein the marker comprises a sensor selected from the group consisting of a radio frequency identification (RFID) sensor and an electrical sensor.

17. The method of claim 14, wherein the marker is configured to be magnetically coupled to the energy device.

18. The method of claim 14, wherein the energy device is configured activate only when the marker is coupled to the energy device.

19. The method of claim 14, wherein the one or more target nerves comprise a nerve selected from a group consisting of a superior ganglia of a vagus nerve, an inferior ganglia of the vagus nerve, a cervical vagus nerve, a thoracic vagus nerve, an abdominal vagus nerve, a celiac plexus nerve, and a splenic nerve.

20. The method of claim 14, where the energy device is configured to monitor a respiration cycle of the patient.

21.-25. (canceled)

26. A system for providing a repeated therapy to an internal organ within a patient, the system comprising:
a marker configured to be affixed to an exterior of the patient; and
an energy device having an energy treatment portion and having a fastener configured to permit securing of the energy device to an exterior of the patient, the energy device further including a mating surface to couple the marker and the energy device, such that when the marker is affixed to the exterior of the patient the energy device can be coupled to the marker allowing for automatic alignment of the energy treatment portion to the internal organ from the exterior of the patient.

27.-38. (canceled)
